(19) United States
(12) Patent Application Publication
(10) Pub. No.: US 2025/0018161 A1
(43) Pub. Date: Jan. 16, 2025

(54) MULTI-NEEDLE MODULE FOR SKIN TREATMENT

(71) Applicants: Hyun Mee NOH, Yongin-si (KR); NOBAMEDI CO., LTD., Yongin-si (KR)

(72) Inventor: Hyun Mee NOH, Yongin-si (KR)

(73) Assignees: Hyun Mee NOH, Yongin-si (KR); NOBAMEDI CO., LTD., Yongin-si (KR)

(21) Appl. No.: 18/509,871

(22) Filed: Nov. 15, 2023

(30) Foreign Application Priority Data
Jul. 14, 2023 (KR) 10-2023-0092077

(51) Int. Cl. A61M 37/00

(52) U.S. Cl. CPC A61M 37/0015; A61M 2037/0023; A61M 2037/0053

(57) ABSTRACT

A multi-needle module for skin treatment is connected to a syringe and mounted on an injection device and includes an upper body open on front and rear sides, and includes side walls, needle pipes formed therein, through which the needles penetrates, and an insertion end formed on an outer periphery of the rear opening, a lower body including an opening formed on a front side, a space defined therein, an inlet hole formed in the bottom, an injection port formed on a rear side, a stepped portion formed on an inner peripheral surface of the front opening to define injection holes to be fitted with insertion end, a connection part inserted into the upper body, and including insertion holders formed on a front side, and needles coupled to each insertion holder, in which injection holes are formed, through which adhesive is injected, and adhesive injection pipes including discharge ports.

### CROSS-REFERENCE TO RELATED APPLICATION

The present application is based upon and claims the benefit of priority to Korean Patent Application No. 10-2023-0092077, filed on Jul. 14, 2023, the disclosure of which is hereby incorporated by reference herein in its entirety.

### TECHNICAL FIELD

The present disclosure relates to a multi-needle module for skin treatment, and more specifically, to a multi-needle module for skin treatment for injecting drugs into skin for effective skin treatment.

### BACKGROUND

In general, the treatment method (hereinafter referred to as ‘multi-hole treatment method’), which involves forming a number of microscopic holes in the skin and allowing drugs, etc. to be injected into the body, is widely used to eliminate various skin problems such as wrinkles, blemishes, stretch marks, acne, freckles, pigmentation, etc., maintain hair health, improve hair loss, treat obesity, and so on.

This multi-hole treatment method can use the self-regenerative power of damaged skin tissue to grow new skin or allow drugs, etc. to penetrate into the body through tiny holes, thereby achieving rapid results.

The related multi-needle module for skin treatment has the problem of requiring additional parts such as vacuum pumps and suction pipes for suctioning the skin, which not only increases high manufacturing costs, but also makes the procedure rather cumbersome for the operator.

To improve this, the present inventor has applied for Korean Patent Application No. 10-2021-0160660, “Multi-needle module for skin treatment.”

In Korean Patent Application No. 10-2021-0160660 mentioned above, the adhesive is injected from the front side of the module and sufficiently injected into the rear side of the module, thereby enabling adhesion between each component member. To improve the needle manufacturing process and increase productivity, the needle part, i.e. the adhesive injection port, is located on the rear side of the needles. However, there is the inconvenience of having to process various cannulas or prepare injection molded products of various lengths to create various exposed lengths of needles.

Meanwhile, the related needle module has a problem in that the process is complex and the adhesion process has to be performed manually by injecting adhesive twice to fix the needle, resulting in low productivity.

As shown in FIG. 9, the multi-needle module for skin treatment is connected to a syringe and mounted on an injection device to inject a multi-needle, and has a problem in that the multi-needle module is not firmly connected to the injection device and the multi-needle module is rotated during the procedure because the procedure is performed at high speed.

### SUMMARY

In order to improve the problems described above, an object of the present disclosure is to provide a multi-needle module for skin treatment, in which adhesive is injected through a rear injection port and evenly injected inside, allowing each component to be bonded, and in which the internal air is discharged to the outside during the injection process so as to ensure that the adhesive can be injected smoothly, and in which the multi-needle module can be firmly fixed without rotating or coming off unexpectedly when it is mounted on the injection device.

It is an object of the present disclosure to provide a multi-needle module for skin treatment, which is connected to a syringe and mounted on an injection device, and may be achieved by a multi-needle module for skin treatment including an upper body, a lower body, a connection part, and needles, in which the upper body may be open on front and rear sides, and may include side walls, a plurality of needle pipes formed therein, through which the needles penetrates, and an insertion end formed on an outer periphery of the rear opening, the lower body may include an opening formed on a front side, side walls formed so as to define a space inside, an inlet hole formed in the bottom, an injection port formed on a rear side, through which a chemical solution is injected, a stepped portion formed on an inner peripheral surface of the front opening, and one or more injection holes formed on the rear side, the insertion end of the upper body may be fitted into the fitting groove of the lower body, so that the insertion end is in close contact with the stepped portion, the connection part may be inserted between the upper body and the lower body, and may include a plurality of insertion holders and needle holes formed on a front side, the front side of the connection part and a rear end of the upper body may be spaced apart from each other to define a receiving space, and adhesive F injected into the injection holes may be filled in the receiving space to adhesively fix the upper body, the lower body, the connection part, and the needles into an integrated structure at once.

The needle pipes include needle holes formed on front ends so that the needles can be inserted therein, and needle through holes formed on rear ends in communication with the needle holes, and the needle through holes may be connected to lattice walls, and filling grooves to be filled with the adhesive may be formed between the lattice walls.

The upper body may include the needle through holes formed on the rear ends of the needle pipes, in which the needle through holes may be connected to the lattice walls, and filling grooves to be filled with the adhesive may be formed between the lattice walls. the connection part includes a plate-shaped base and a insertion holder protruding from an outer surface of the base, in which the insertion holder is inserted into the filling groove, and an end of the insertion holder is supported in contact with the end of the filling groove, and the adhesive injected into the injection holes may be filled in the receiving space and spaces between the filling grooves and the insertion holders to adhesively fix the upper body, the lower body, the connection part, and the needles into an integrated structure at once.

The lower body may include distribution passages formed on the front side communicating with the inlet hole, and an injection port formed on the rear side communicating with the inlet hole, the injection port may include a passage formed inside, a flange formed on an outer peripheral surface, and an upper injection port and a lower injection port based on the flange, and the lower injection port may include a port coupling hole formed at one end, and the upper injection port may include a rotation prevention part.

The connection part may include a base that is formed in a plate shape and that includes a support part protruding to form a stepped portion, and insertion holders protruding from an outer surface of the base.

The insertion holder may include holder support members formed outside the insertion holders.

The rotation prevention part may include, on both sides thereof, straight parts having vertical surfaces.

The injection part includes first and second injection holes, each of which is formed on a rear side surface of the lower body 200, respectively, to have a positional deviation.

The lower body may include, formed therein, adhesive injection pipes including discharge ports which communicate with the injection holes and through which the adhesive is moved, the connection part may include grooves to be connected to the adhesive injection pipes so as to form pipes, and the upper body may include filling grooves to be filled with the adhesive discharged into the grooves, and the adhesive introduced into the injection holes may be moved to the adhesive injection pipes and the grooves and fills the receiving space and the filling grooves so as to adhesively fix the upper body, the lower body, the connection part, and the needles into an integrated structure at once.

According to the present disclosure, adhesive is injected through the rear injection ports and evenly injected inside, allowing each component to be bonded, and in which the internal air is discharged to the outside during the injection process so as to ensure that the adhesive can be injected smoothly, and in which the multi-needle module can be firmly fixed without rotating or coming off unexpectedly when it is mounted on the injection device.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 is a front perspective view of a multi-needle module for skin treatment according to the present disclosure;

FIG. 2 is a rear perspective view of the multi-needle module for skin treatment according to the present disclosure;

FIG. 3 is an exploded front perspective view of the multi-needle module for skin treatment according to the present disclosure;

FIG. 4 is an exploded rear perspective view of the multi-needle module for skin treatment according to the present disclosure;

FIG. 5A is a cross-sectional perspective view of the multi-needle module for skin treatment in assembled state according to the present disclosure;

FIG. 5B is a cross-sectional view of the multi-needle module for skin treatment in assembled state according to the present disclosure;

FIG. 6 is a cross-sectional perspective view of the multi-needle module for skin treatment on a lower body in an injection device according to the present disclosure;

FIG. 7 is a cross-sectional view showing a state in which adhesive is injected into the multi-needle module for skin treatment according to the present disclosure;

FIG. 8 is a perspective view of a multi-needle module for skin treatment according to another embodiment of the present disclosure;

FIG. 9 is a perspective view of a multi-needle module for skin treatment according to still another embodiment of the present disclosure;

FIG. 10 is a perspective view of a multi-needle module for skin treatment according to an embodiment of the present disclosure; and

FIG. 11 is a view showing a state in which the multi-needle module for skin treatment according to the present disclosure is mounted on an injection device for use.

### DETAILED DESCRIPTION

Hereinafter, the preferred embodiment will be described in detail below based on the attached drawings.

The examples to be described below are intended to explain the present disclosure in detail such that a person skilled in the art can easily practice the present disclosure, and do not mean that the technical idea and scope of the disclosure are limited.

In addition, it is to be noted that the size or shape of the components shown in the drawings may be exaggerated for clarity and convenience of explanation, and terms specifically defined in consideration of the configuration and operation of the present disclosure may vary according to the intention or custom of the user or operator, and these terms should be defined based on the content throughout this specification.

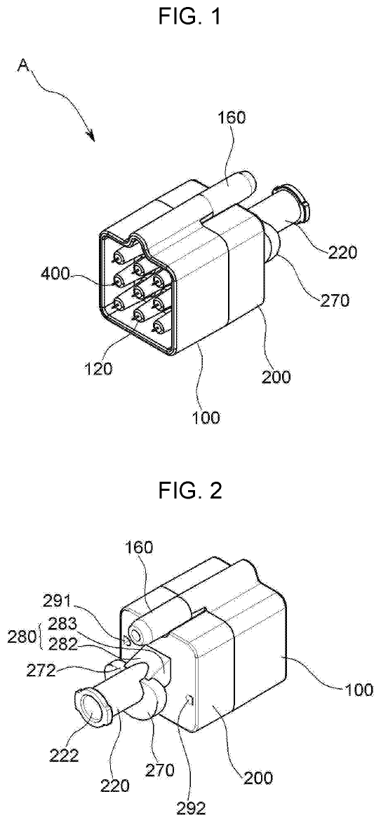

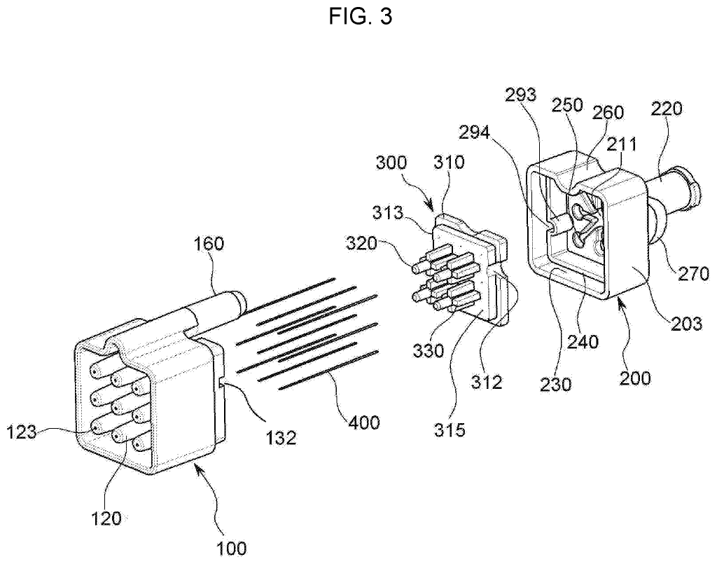

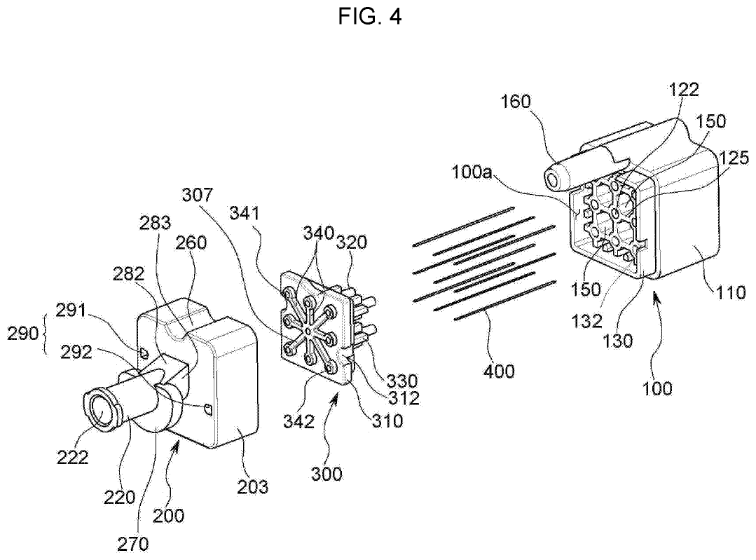

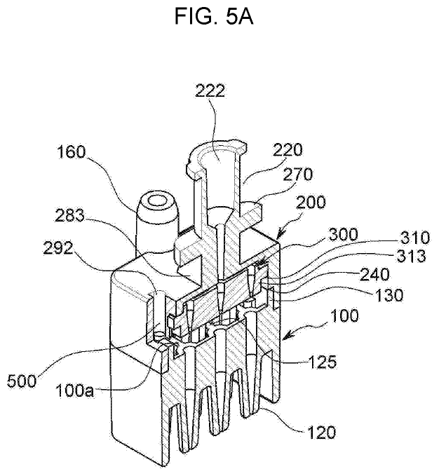

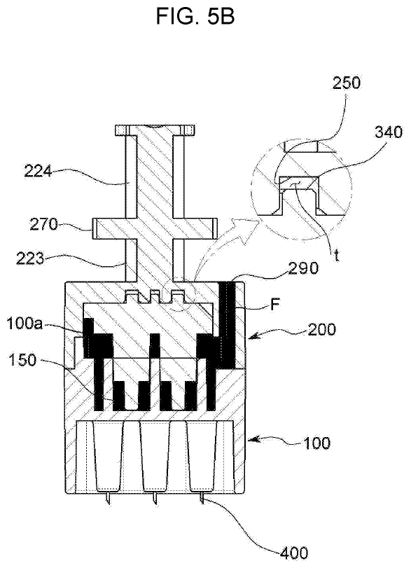

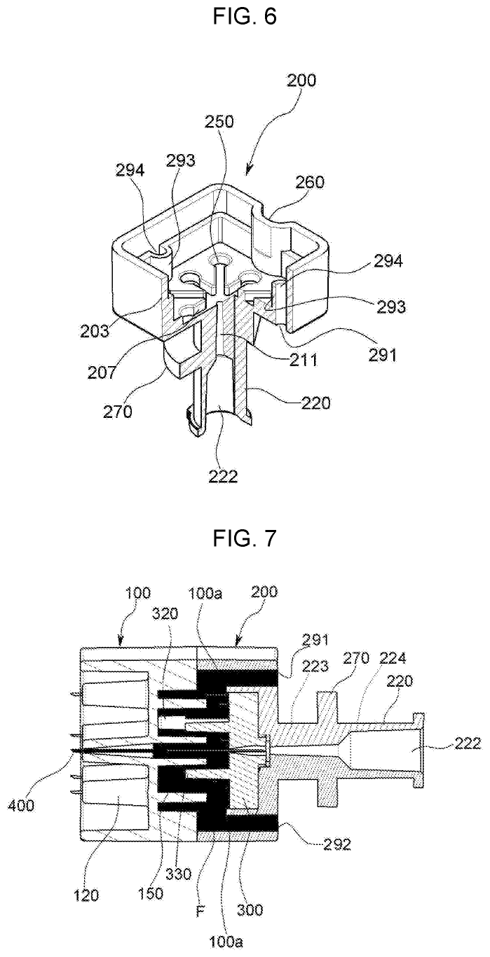

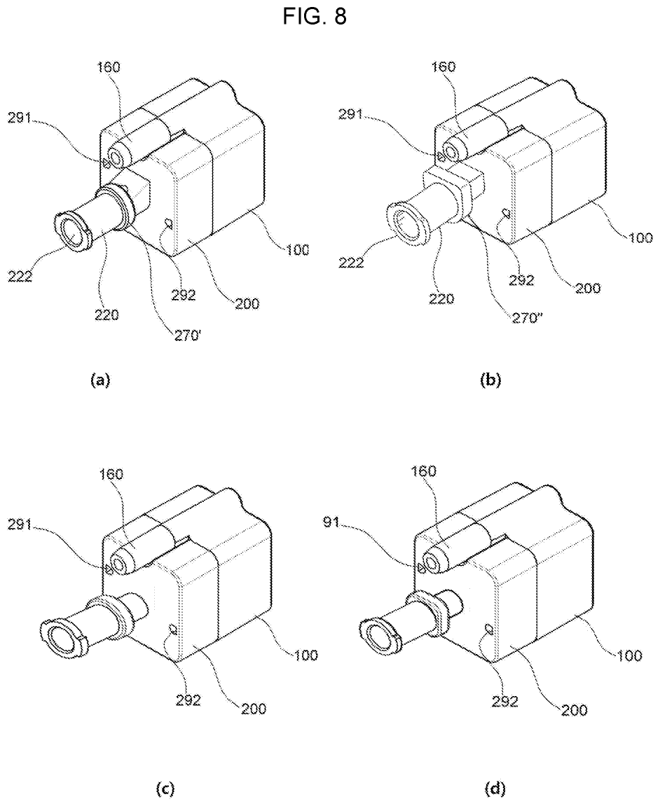

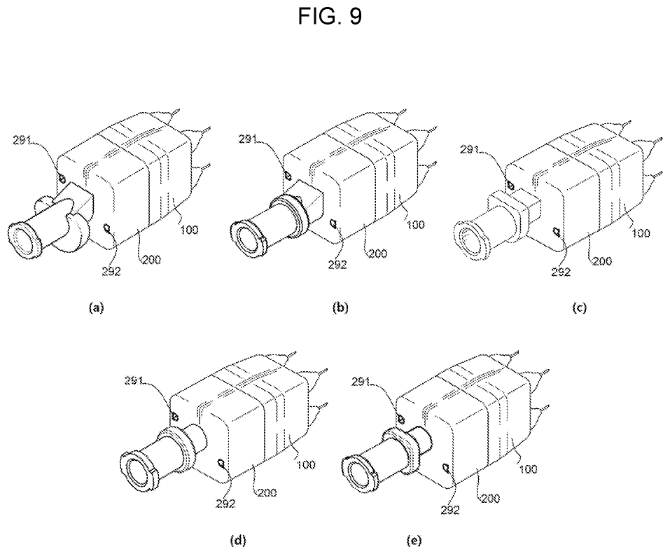

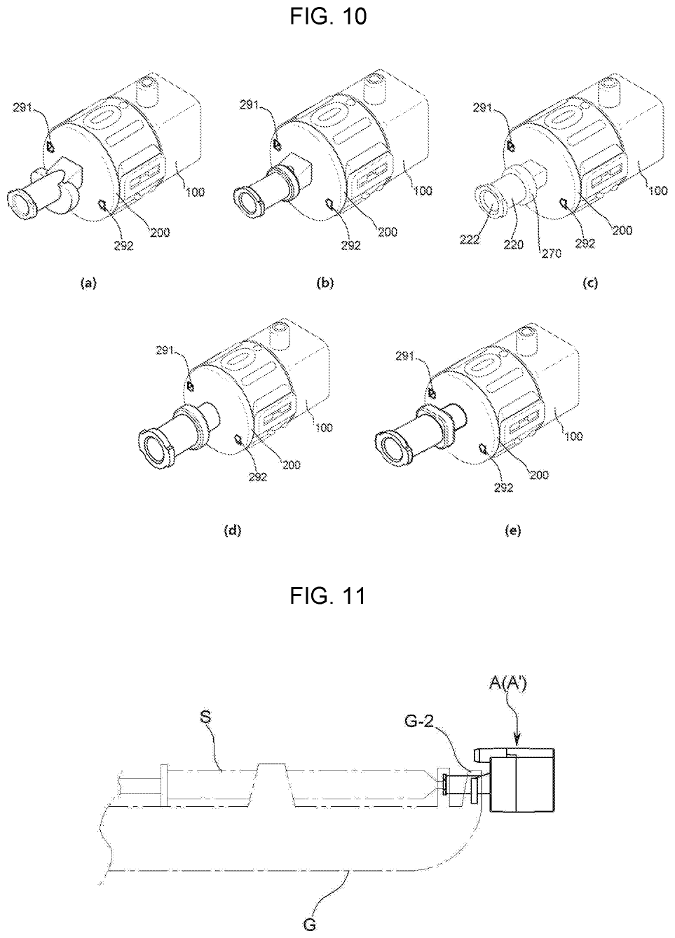

In the accompanying drawings, FIG. 1 is a front perspective view of a multi-needle module for skin treatment according to the present disclosure, FIG. 2 is a rear perspective view of the multi-needle module for skin treatment according to the present disclosure, FIG. 3 is an exploded front perspective view of the multi-needle module for skin treatment according to the present disclosure, FIG. 4 is an exploded rear perspective view of the multi-needle module for skin treatment according to the present disclosure, FIG. 5A is a cross-sectional perspective view of the multi-needle module for skin treatment in assembled state according to the present disclosure, FIG. 5B is a cross-sectional view of the multi-needle module for skin treatment in assembled state according to the present disclosure, FIG. 6 is a cross-sectional perspective view of the multi-needle module for skin treatment on a lower body in an injection device according to the present disclosure, FIG. 7 is a cross-sectional view showing a state in which adhesive is injected into the multi-needle module for skin treatment according to the present disclosure, FIG. 8 is a perspective view of a multi-needle module for skin treatment according to another embodiment of the present disclosure, FIG. 9 is a perspective view of a multi-needle module for skin treatment according to still another embodiment of the present disclosure, FIG. 10 is a perspective view of a multi-needle module for skin treatment according to an embodiment of the present disclosure, and FIG. 11 is a view showing a state in which the multi-needle module for skin treatment according to the present disclosure is mounted on an injection device for use.

First, referring to FIGS. 1 to 11, a multi-needle module A for skin treatment according to the present disclosure is connected to a syringe S and mounted on an injection device G, and includes, as main parts, a needle 400, an upper body 100, a lower body 200, and a connection part 300.

The needle 400 includes one or more needles, and in this embodiment, a multi-needle module having nine needles is illustrated. Of course, the number of needles need not be limited thereto.

The upper body 100 is open on front and rear sides, and includes side walls 110, a plurality of needle pipes 120 formed therein, through which the needles 400 penetrate, and an insertion end 130 formed on an outer periphery of the rear opening. The front end of the upper body 100 comes into contact with the skin.

The lower body 200 includes an opening formed on a front side, a space formed therein, an inlet hole 211 formed on the bottom, and an injection port 220 on the rear side, which is connected to the syringe so as to inject a chemical solution therethrough. The lower body 200 is combined with the upper body 100. A stepped portion 240 is formed on an inner peripheral surface of the front opening, and one or more injection holes 290 are formed on the rear side.

The connection part 300 is inserted between the upper body 100 and the lower body 200, and a plurality of insertion holders 320 and a plurality of needle holes 315 are formed on a front side, and the needles 400 are penetrated through and fastened in the insertion holders 315. The insertion holder 320 may be formed in a cylindrical shape, and a holder support member 330 is formed outside the insertion holder 320.

On a rear side of the connection part 300, there are distribution holes 342 formed corresponding to the needle holes 315. A distribution hole stepped portion 340 is formed and protruding around the distribution hole 342. The needle 400 is connected through the needle hole 315 and the distribution hole 342.

A front side of the connection part 300 and an rear end of the upper body 100 are spaced apart from each other to define a receiving space 100a, and adhesive F fed into the injection holes 290 is filled in the receiving space 100a to adhesively fix the upper body 100, the lower body 200, the connection part 300, and the needle 400 into an integrated structure at once.

The one or more injection holes 290 are formed on a rear side of the lower body 200 so that the adhesive F is injected therethrough, and an adhesive injection pipe 293 having a discharge port 294 which communicates with the injection holes 290 and through which the adhesive is moved, is formed on an inner peripheral surface of the lower body 200.

Referring to FIGS. 3 and 4, the injection holes 290 includes first and second injection holes 291 and 292 formed on the rear side of the lower body 200 and symmetrically formed on both sides of the injection port 220, and the adhesive injection pipes 293 that communicates with the first and second injection holes 291 and 292 and are formed on both sides of the inner peripheral surface of the lower body 200, respectively.

There are one or more injection holes 290 formed, and preferably, two injection holes 291 and 292 are formed. When the adhesive F is injected into the first injection hole 291, the air filled in the receiving space 100a is discharged through the second injection hole 292. Then, the adhesive F is injected into the first injection hole 291 and it can be confirmed whether the adhesive F is filled into the second injection hole 292.

The first injection hole 291 and the second injection hole 292 may be positioned to optimize the flow of air described above, the first injection hole 291 may be formed on the outside of the lower body 200, avoiding the location of a distribution passage 250, so as not to interfere with the location of the distribution passage 250 formed on the bottom surface of the lower body 200, and the second injection hole 292 may be formed to have a positional deviation so as to be spaced apart as much as possible from the first injection hole 291.

The connection part 300 includes a plate-shaped base 310 and the insertion holders 320 protruding from the outer surface of the base 310, and grooves 312 formed on both sides of the connection part 300 to be connected to the adhesive injection pipes 293 to form pipes through which the adhesive F is injected.

The insertion holders 320 are inserted into filling grooves 150, and ends of the insertion holders 320 are supported in contact with ends of the filling grooves 150.

Needle through holes 122 are formed inside the upper body 100, and are formed on rear ends of the needle pipes 120 to be filled with the adhesive discharged from the discharge ports 294 of the adhesive injection pipe 293, and the needle through holes 122 are connected to lattice walls 125, and the filling grooves 150 to be filled with the adhesive are formed between the lattice walls 125.

Therefore, the adhesive F injected into the injection holes 290 is filled in the receiving space 100a and in the spaces between the filling grooves 150 and the insertion holders 320 to adhesively fix the upper body, the lower body, the connection part, and the needles into an integrated structure at once.

Referring to FIGS. 2 and 4, the first and second injection holes 291 and 292 are formed at upper and lower portions of the rear side surface of the lower body 200, respectively, to have a positional deviation.

As an example, with respect to the injection port 220 as a reference, the first injection hole 291 may be formed in an upper left corner and the second injection hole 292 may be formed in a lower right corner.

Due to the positional deviation of the first and second injection holes 291 and 292, the adhesive F can be sufficiently injected without leaving any empty space inside, because, for example, the adhesive F injected into the first injection hole 291 can be discharged into the second injection hole 292 after filling the entire internal space.

Referring to FIGS. 4 and 5B, it is preferable that the first and second injection holes 291 and 292 are formed symmetrically around an outer periphery of the base 310 to avoid a plurality of distribution hole stepped portions 340 of the connection part 300, and to have upper and lower positional deviations, so that the adhesive F is positioned so as not to interfere with the distribution hole stepped portions 340 through which the chemical solution passes.

When the adhesive F is injected through the first injection hole 291, the adhesive F may be injected into the receiving space 100a formed inside between the lower body 200 and the upper body 100, that is, between the rear side of the connection part 300 and the rear end of the upper body 100 spaced apart from each other, and the air inside the receiving space 100a may be discharged through the second injection hole 292, thereby allowing the adhesive F to be fully injected.

The upper body 100 includes the needle through holes 122 formed on the rear ends of the needle pipes 120 to allow the needle 400 to be inserted, and the needle through holes 122 are connected to the lattice walls 125, and the filling grooves 150 to be filled with adhesive are formed between the lattice walls 125.

Inside each needle pipe 120, a needle hole 123 is formed on a front end and the needle through hole 122 is formed on a rear end such that the needle 400 can be inserted, so that the needle 400 may be passed through the needle pipe 120 and positioned. The needle pipe 120 may be formed in a cylindrical shape, and an outer end of the needle hole 123 may have a flat cross-section to contact the skin, and when the needle 400 is inserted into the skin, the flat cross-section pressurizes the surrounding skin, making it easier for the needle 400 to puncture the skin, thus facilitating injection of the chemical solution into the skin.

Referring to FIGS. 4, 5A, and 7, the upper body 100 is formed such that the rear ends of the plurality of needle pipes 120 and the insertion end 130 have a height difference, and the lattice walls 125 on the rear ends of the needle pipes 120 are formed lower than the insertion end 130. The insertion end 130 of the upper body 100 is fitted into the fitting groove 230 of the lower body 200, the insertion end 130 is in close contact with the stepped portion 240, and a space is formed between the upper body 100 and the lower body 200. The connection part 300 is positioned between the upper body 100 and the lower body 200, the insertion holders 320 are inserted into the filling grooves 150 of the upper body 100, the ends of the insertion holders 320 are supported in contact with the ends of the filling grooves 150, spaces are formed between the filling grooves 150, the insertion holders 320, and the holder support members 330, and the adhesive F is injected into the spaces.

The insertion end 130 may be formed on the outer periphery of the rear opening of the upper body 100, and insertion end groove 132 may be formed in the insertion end 130 at positions corresponding to the grooves 312 formed on both sides of the connection part 300. The insertion end grooves 132 facilitate the injection of the adhesive F into the adhesive injection pipes 293.

The insertion holder 320 may be formed in a polygonal pillar shape including, for example, cone shape, or cylinder shape, but is not limited thereto. The holder support members 330 are formed outside the insertion holders 320. The holder support member 330 may be formed to have a width so that that it 330 can be inserted into the filling groove 150, include a space where the adhesive F may be injected so as to adhesively fix the upper body 100 and the connection part 300 securely, and also include the holder support members 330 so as to reduce the amount of adhesive F used. In addition, the holder support member 330 may be formed in a radial polygonal pillar shape on the outside of the insertion holder 320 to increase a bonding area with the adhesive F.

The lower body 200 includes the stepped portion 240 formed on the inner peripheral surface, which communicates with the opening and includes the fitting groove 230 to be fitted with the insertion end 130 of the upper body 100.

The upper body 100 may include a suction pipe connector 160 to which a suction pipe is connected for use, and if the suction pipe connector 160 is formed, a recess 260 may be formed concavely such that the suction pipe connector 160 of the upper body 100 can be inserted on an outer surface of side walls 203 of the lower body 200.

A suction part (not shown) may be connected to the suction pipe connector 160, which may, during the injection process of the needles 400, cause the multi-needle module A for skin treatment to be brought into contact with the skin with the outer peripheral surface of the upper body 100 being in contact with the skin, such that negative pressure is generated in the space between the skin and the needle pipe 120 of the upper body 100, pulling the skin inside the outer peripheral surface of the upper body 100, thereby helping the needles 400 penetrate the skin.

The lower body 200 includes the opening formed on the front side, the side walls 203 formed so as to define a space inside, the distribution passages 250 formed on the bottom, and the injection port 220 formed on the rear surface through which the chemical solution flows through the distribution passages 250. The distribution passages 250 are sunk downward the lower body 200, and the needles 400 are inserted into the distribution passages 250. The distribution passages 250 are connected to the inlet holes 211, and the chemical solution injected through the inlet holes 211 is moved to the distribution passages 250 and to the needles 400 connected to the distribution passages 250.

The distribution passages 250 correspond in number to the needles 400, and are all connected to the central inlet hole 211 to allow the chemical solution to pass through. As shown in FIG. 6, when the needles 400 are 9 pins, the distribution passages 250 include 9 holes, 1 in the center and 8 on the edges, and each distribution passage 250 is connected to eight passages 207 connected to the inlet hole 211.

The plurality of needle holes 315, through which the needles 400 are passed, are formed on the front side of the connection part 300, and the distribution holes 342 corresponding to the needle holes 315 are formed on the rear side of the connection part 300. The distribution hole stepped portions 340 protrude around the distribution holes 342. The needles 400 are passed through the needle holes 315 and the distribution holes 342 and connected.

Chemical solution injection holes 341, which are connected to the inlet holes 211, are formed on the rear side of the connection part 300, and the chemical solution injected through the inlet holes 211 is moved to the distribution passages 250 and then moved to the chemical solution injection holes 341 connected to the distribution passages 250. The chemical solution moved to the chemical solution injection hole 341 is supplied to the needles 400 through the distribution holes 342.

The connection part 300 includes the base 310 including a support part 313 formed in a plate shaped and protruding to form a stepped portion, and the insertion holder 320 protruding from the rear side of the base 310 and the holder support members 330 formed outside the insertion holders 320, in which the insertion holders 320 and the holder support members 330 are inserted into the filling grooves 150 of the upper body 100.

The support part 313 is formed to have a smaller area than the base 310, so that a space into which the adhesive F can be injected is formed between an outer periphery of the support part 313 and an inner peripheral surface of the side of the lower body 200, and the adhesive F is injected into the space such that the connection part 300, the upper body 100, the needles 400, and the lower body 200 are adhesively fixed to an integrated structure at once.

The holder support member 330 is formed in the shape of a radial polygonal column to reinforce the connection area between the insertion holder 320 and the base 310 thereby preventing damages, is hardly moved in the filling grooves 150 and thus positioned stably, and has a wider adhesion area with the adhesive F so as to be securely and adhesively fixed.

Referring to FIGS. 5A and 5B, the plurality of distribution hole stepped portions 340 are formed on the rear side of the connection part 300, and the distribution holes 342 are formed on the ends of each distribution hole stepped portion 340 such that the chemical solution can be injected.

As shown in FIG. 5B, the distribution hole stepped portions 340 are inserted into the distribution passages 250 formed on the bottom surface of the lower body 200, and the distribution hole stepped portions 340 and the distribution passages 250 are spaced apart to form a gap t therebetween, so that the chemical solution can flow through the gap t, and the passages 207 of the distribution passages 250 of the lower body and passages 307 of the distribution holes 342 of the connection part 300 are formed to correspond to each other, and the distribution passages 250 and the distribution hole stepped portions 340 come into contact with each other to form passages through which the chemical solution is moved, and the chemical solution is supplied to the needles 400 through the passages.

The distribution holes 342 correspond in number to the needles 400, and are all connected to the central chemical solution injection holes 341. As shown in FIG. 4, when the needles 400 are 9 pins, there are 8 distribution holes 342 on the edges and one chemical solution injection hole 341 formed in the center, making a total of 9 holes, and the chemical solution injection holes 341, which also perform the function of the distribution holes 342, are coupled with the needles 400, and each distribution hole 342 is connected to the eight passages 307 leading to the chemical solution injection holes 341.

The injection port 220 includes a port coupling hole 222 formed at one end, a passage formed inside, and a flange 270 formed on an outer peripheral surface of the injection port 220 extending in the longitudinal direction. The flange 270 is inserted into and coupled to a fastener G-2 of the handpiece G to be fixed, and the flange 270 is formed in a shape corresponding to the fastener G-2.

The injection port 220 is divided into upper and lower parts based on the flange 270, and the injection port 220 between the rear side of the lower body 200 and the flange 270 is formed as an upper injection port 223, and the injection port 220 below the flange 270 is formed as a lower injection port 224.

The lower injection port 223 has a cylindrical tubular shape, and the upper injection port 223 has a rotation prevention part 280 formed by deforming the cylindrical tubular shape. The rotation prevention part 280 includes straight parts 283 formed vertically on both sides such that, during the procedure in which the multi-needle module A for skin treatment is connected to the syringe S and mounted on the injection device G, these straight parts 283 on both sides make the multi-needle module A for skin treatment firmly connected to the injection device G. The rotation prevention part 280 may additionally include a flat plate part 282 inclined between the straight parts 283.

The flange 270 has an oval plate shape, and includes a cut surface 272 formed on a portion and connected to the straight part 283 of the rotation prevention part 280.

Alternatively, as shown in FIGS. 8 to 10, the flange 270′ has a round jaw shape that protrudes in a circular shape, or the flange 270″ has a round jaw shape that protrudes in a square shape. The flange 270 may be formed in various shapes, and the flange 270 may be formed in various shapes corresponding to the fastener G-2. Alternatively, the flange 270 is formed in a round jaw shape protruding in a U shape with one side being straight. Therefore, when the flange 270 is mounted on the handpiece G, and the cut surface 272 of the flange 270 is coupled to the fastener G-2 of the handpiece G, the multi-needle module A for skin treatment of the present disclosure can be fixed and prevented from rotating.

Alternatively, the upper injection port 223 includes the rotation prevention part 280, and the rotation prevention part 280 includes straight parts 283 having vertical surfaces on both sides. Therefore, when the flange 270 is mounted on the handpiece G, and the cut surface 272 of the flange 270 is coupled to the fastener G-2 of the handpiece G and firmly fastened to the straight part 283 of the rotation prevention part 280, the multi-needle module A for skin treatment of the present disclosure can be fixed and prevented from rotating. The longer the straight part 283 is formed, the more firmly it may be fixed. That is, a stepped portion is generated between the injection port 220 and the straight part 283, and the longer the length of the cut surface of the straight part 283, the more firmly it may be fixed.

The process of injecting the adhesive F into the multi-needle module A for skin treatment configured as described above according to the present disclosure will be described below.

The assembled multi-needle module A for skin treatment is placed upside down on the jig such that the upper body 100 is seated on the upper surface of the jig.

Then, an injection device (not shown) is connected so as to be connected to the first and second injection holes 291 and 292 of the lower body 200.

Then, adhesive is injected into one of the first and second injection holes 291 and 292. A preset amount is injected for a set injection time.

Then, the adhesive F is cured using a curing machine.

Therefore, as shown in FIG. 7, by filling the internal space with adhesive F (shown in black), the internal needle 400, the connection part 300, the lower body 200, and the upper body 100 may be integrated.

Although the description has been made in relation to the preferred embodiment, it will be readily apparent to those skilled in the art that various modifications and variations can be made without departing from the gist and scope of the disclosure, and it is obvious that all such changes and modifications fall within the scope of the appended claims.

1. A multi-needle module for skin treatment, which is connected to a syringe and mounted on an injection device and comprises:
an upper body, a lower body, a connection part, and needles, wherein
the upper body is open on front and rear sides, and includes a side wall, a plurality of needle pipes formed therein, through which the needles penetrates, and an insertion end formed on an outer periphery of the rear opening,
the lower body includes an opening formed on a front side, side walls formed so as to define a space inside, an inlet hole formed in the bottom, an injection port formed on a rear side, through which a chemical solution is injected, a stepped portion formed on an inner peripheral surface of the front opening, and one or more injection holes formed on the rear side,
the insertion end of the upper body is fitted into the fitting groove of the lower body, so that the insertion end is in close contact with the stepped portion,
the connection part is inserted between the upper body and the lower body, and includes a plurality of insertion holders and needle holes formed on a front side,
the front side of the connection part and a rear end of the upper body are spaced apart from each other to define a receiving space, and
adhesive F injected into the injection holes is filled in the receiving space to adhesively fix the upper body, the lower body, the connection part, and the needles into an integrated structure at once.

2. The multi-needle module for skin treatment according to claim 1, wherein the needle pipes include needle holes formed on front ends so that the needles can be inserted therein, and needle through holes formed on rear ends in communication with the needle holes, and
the needle through holes are connected to lattice walls, and filling grooves are formed between the lattice walls to be filled with the adhesive.

3. The multi-needle module for skin treatment according to claim 1, wherein
the upper body includes needle through holes formed on the rear ends of the needle pipes,
the needle through holes are connected to lattice walls, and filling grooves are formed between the lattice walls to be filled with the adhesive,
the connection part includes a plate-shaped base and insertion holders protruding from an outer surface of the base,
the insertion holders are inserted into the filling grooves, and ends of the insertion holders are supported in contact with the ends of the filling grooves, and
the adhesive F introduced into the injection holes is filled in the receiving space and spaces between the filling grooves and the insertion holders so as to adhesively fix the upper body, the lower body, the connection part, and the needles into an integrated structure at once.

4. The multi-needle module for skin treatment according to claim 1, wherein
the lower body includes distribution passages formed on the front side communicating with the inlet hole, and an injection port formed on the rear side communicating with the inlet hole,
the injection port includes a passage formed inside, a flange formed on an outer peripheral surface, and an upper injection port and a lower injection port based on the flange,
the lower injection port has a port coupling hole formed at one end, and
the upper injection port includes a rotation prevention part.

5. The multi-needle module for skin treatment according to claim 1, wherein the connection part includes a base that is formed in a plate shape and that includes a support part protruding to form a stepped portion, and insertion holders protruding from an outer surface of the base.

6. The multi-needle module for skin treatment according to claim 1, wherein the insertion holders include holder support members formed outside the insertion holders.

7. The multi-needle module for skin treatment according to claim 4, wherein the rotation prevention part includes, on both sides thereof, straight parts having vertical surfaces.

8. The multi-needle module for skin treatment according to claim 1, wherein the injection holes include first and second injection holes, each formed on the rear side of lower body to have a positional deviation.

9. The multi-needle module for skin treatment according to claim 1, wherein
the lower body includes adhesive injection pipes formed therein and including discharge ports which communicate with the injection holes and through which the adhesive F is moved,
the connection part includes grooves connected to the adhesive injection pipes to form pipes,
the upper body includes filling grooves formed so as to be filled with the adhesive F discharged into the grooves, and
the adhesive F introduced into the injection holes is moved to the adhesive injection pipes and the grooves and fills the receiving space 100a and the filling grooves so as to adhesively fix the upper body, the lower body, the connection part, and the needles into an integrated structure at once.
